(19) United States
(12) Patent Application Publication
(10) Pub. No.: US 2021/0169839 A1
(43) Pub. Date: Jun. 10, 2021

(54) PEDIATRIC IMMEDIATE-RELEASE FORMULATION OF THE POTASSIUM CHANNEL OPENER EZOGABINE

(71) Applicant: Xenon Pharmaceuticals Inc., Burnaby (CA)

(72) Inventors: Jean-Jacques Alexandre Cadieux, Burnaby (CA); Matthew David Tandy, Vancouver (CA)

(21) Appl. No.: 17/109,937

(22) Filed: Dec. 2, 2020

Related U.S. Application Data
(60) Provisional application No. 62/942,579, filed on Dec. 2, 2019.

(51) Int. Cl. A61K 31/27; A61K 9/00; A61K 47/38; A61K 47/36; A61K 47/10; A61K 47/32

(52) U.S. Cl. CPC A61K 31/27; A61K 9/0053; A61K 47/32; A61K 47/36; A61K 47/10; A61K 47/38

(57) ABSTRACT

Disclosed herein are immediate-release oral pharmaceutical formulations comprising a therapeutically effective amount of ezogabine and one or more pharmaceutically acceptable excipients for use in treating epilepsy and/or epileptic seizure disorders in a mammal, preferably a human, more preferably a child. Also disclosed are methods of using and making the immediate-release oral pharmaceutical formulations.

### FIELD OF THE INVENTION

The present disclosure is directed to immediate-release pharmaceutical formulations for oral administration to a mammal, preferably a human, more preferably a child, comprising one or more pharmaceutically acceptable excipients and a therapeutically effective amount of the potassium channel opener, ezogabine. In particular, the present disclosure is directed to such immediate-release oral pharmaceutical formulations for treating epilepsy and/or epileptic seizure disorders in children, particularly for KCNQ2-related neonatal developmental and epileptic encephalopathy (KCNQ2-DEE).

### BACKGROUND OF THE INVENTION

KCNQ2-related neonatal developmental and epileptic encephalopathy (KCNQ2-DEE), otherwise known as EIEE7, is a rare and severe neurodevelopmental disorder in infants and children with a significant seizure burden and profound developmental impairment. KCNQ2-DEE is uniquely characterized by multiple, daily, refractory seizures presenting within the first week of life with a prominent tonic component and autonomic signs. Seizures are often accompanied by clonic jerking or complex motor behavior. An electroencephalogram (EEG) at onset of the disease shows a burst suppression pattern later evolving into multifocal epileptiform activity. The infants afflicted with KCNQ2-DEE usually develop a severe to profound intellectual disability with axial hypotonia which can be accompanied by limb spasticity. The seizure activity typically decreases with age with patients often becoming seizure free or experiencing more minor seizure burden by 3 to 5 years of age; however, thereafter seizures can reoccur in clusters. The intellectual disability and other co-morbidities are not reversed or improved with age and patients generally require life-long care. Patients are often non-verbal and some children may also have autistic features. Seizure-related bradycardia and oxygen desaturation with cyanosis have been observed, and are thought to contribute to the significant risk of Sudden Unexpected Death in Epilepsy, or SUDEP, in these children. KCNQ2-DEE is rare, representing around 10% of patients with epileptic encephalopathy with onset in the first three months of life; however, the incidence of KCNQ2-DEE is approximately 2.8/100,000 live births, which is roughly half the number of births of Dravet Syndrome, the most common genetic type of early infantile epileptic encephalopathy.

Ezogabine (also known as retigabine) is a known neuronal KCNQ (Kv7) potassium channel opener and has the following structure:

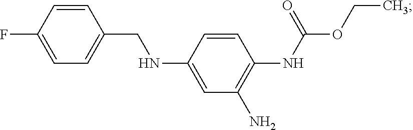 [SMILES: CCOC(=O)Nc1ccc(NCc2ccc(F)cc2)cc1N]

and has a chemical name of 2-amino-4-(4-fluorobenzylamino)-1-ethoxycarbonylaminobenzene. Ezogabine, its preparation and its use as an anti-epileptic is disclosed in U.S. Pat. No. 5,384,330.

Ezogabine was first identified as an analogue of the analgesic compound flupirtine in the late 1980s. Ezogabine demonstrated broad spectrum activity in studies designed to identify novel anti-convulsant agents using a battery of rodent seizure models (see Kupferberg, H., Epilepsia (1989), 30 (Suppl. 1):551-556). Ezogabine was approved for partial onset seizures in 2011 and marketed by GlaxoSmithKline as a coated immediate-release tablet formulation (Potiga®/Trobalt™) for adjunctive treatment of focal seizures in patients aged 18 years and older, but was removed from the market in 2017 for commercial reasons following black-box warnings related to discoloration of skin, lips, nails and retinal pigmentary changes. These discoloration instances appear to be related to formation of chromophoric ezogabine dimers after long term use (Prescott, J. S. and Evans, C. A., “Pigmentary abnormalities (discoloration) associated with ezogabine/retigabine treatment: nonclinical aspects”, Poster 2.324 presented at the 68th Annual Meeting of the American Epilepsy Society (AES), Seattle, Wash., U.S.A., Dec. 5-9, 2014).

While the tablet formulation was used off-label in the KCNQ2-DEE pediatric population (see Millichap, J. J. et al., Neurol. Genet., October 2016, 2:1-5), a pediatric formulation was not marketed.

While significant advances have been made in treating KCNQ2-DEE in children, there remains a substantial need for improved formulations of ezogabine for the treatment of KCNQ2-DEE in children.

### SUMMARY OF THE INVENTION

In some embodiments, the present disclosure is directed to immediate-release pharmaceutical formulations for oral administration to a mammal, preferably a human, more preferably a child, comprising one or more pharmaceutically acceptable excipients and a therapeutically effective amount of the potassium channel opener, ezogabine. In one embodiment, the present disclosure is directed to such immediate-release formulations for treating epilepsy and/or epileptic seizure disorders in children, particularly for KCNQ2-related neonatal developmental and epileptic encephalopathy (KCNQ2-DEE).

In one embodiment, the present disclosure is directed to immediate-release oral pharmaceutical formulations comprising one or more pharmaceutically acceptable excipients and a therapeutically effective amount of ezogabine, wherein the formulations allow for flexible weight-based dosing without requiring extemporaneous compounding of the formulations prior to oral administration to a mammal, preferably a human, more preferably a child.

In another embodiment, the present disclosure is directed to a method of treating epilepsy and/or epileptic seizure disorders in a mammal, preferably in humans, more preferably in children, particularly to methods of treating KCNQ2-related neonatal developmental and epileptic encephalopathy (KCNQ2-DEE) in children, wherein the method comprises administering a therapeutically effective amount of an immediate-release oral pharmaceutical formulation disclosed herein to a child in need thereof.

In another embodiment, the present disclosure is directed to a method of preparing immediate-release oral pharmaceutical compositions comprising one or more pharmaceutically acceptable excipients and a therapeutically effective amount of ezogabine.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1 provides the dissolution profile of an immediate-release oral pharmaceutical formulation of the invention (Example #12).

FIG. 2 provides the plasma concentration of ezogabine after oral administration of an immediate-release oral pharmaceutical formulation of the invention (Example #12) and crushed Potiga (powdered ezogabine tablets) in rats.

FIG. 3 provides the plasma concentration of ezogabine as a function of time following oral administration of a single 400 mg dose of ezogabine (as 2.0 g of an immediate-release oral pharmaceutical formulation of the invention (Example #12)) under fasted or fed conditions.

### DETAILED DESCRIPTION OF THE INVENTION

The present disclosure is directed to immediate-release oral pharmaceutical formulations comprising one or more pharmaceutically acceptable excipients and a therapeutically effective amount of ezogabine, wherein the immediate-release oral pharmaceutical formulations allow for flexible weight-based dosing without requiring extemporaneous compounding of the formulations prior to oral administration to a mammal, preferably a human, more preferably a child.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to”. Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, the term “about” as used herein means ±20% of the stated value, and in more specific embodiments means ±10%, ±5%, ±2%, and ±1% of the stated value.

### Definitions

Unless defined otherwise in the specification, the following terms and phrases shall have the following meaning:

“API” or “Active Pharmaceutical Ingredient” as used herein refers to ezogabine.

“Immediate-release” as used herein refers to pharmaceutical formulations which disintegrate rapidly upon oral administration to a patient in need thereof and get dissolved to release the active pharmaceutical ingredient (API). Immediate-release may be provided for by way of appropriate pharmaceutically acceptable excipients, which excipients do not prolong, to an appreciable extent, the rate of API release and/or absorption.

The expression “% w/w” refers to a percentage by weight compared to the total weight of the composition being considered.

The expression “% w/v” refers to a weight of a solute in a given volume of solvent. For example, 50% w/v of HPMC is 50 grams of HPMC in 100 mL solvent.

“Mammal” includes humans and both domestic animals such as laboratory animals and household pets, (e.g., cats, dogs, swine, cattle, sheep, goats, horses, and rabbits), and non-domestic animals such as wildlife and the like. In some embodiments, a mammal is a human, preferably a child.

“Child” or “children” as used herein refers to a human child between the ages of birth to about 12 years old having a body weight of between about 2 kg and about 20 kg, and includes a human child younger than 1 month (“neonate”), a human child of 1 month to 24 months (“infant”) and a human child of 2 years to 12 years (“child”).

### Pharmaceutically Acceptable Excipients

In some embodiments, the present disclosure is directed to immediate-release oral pharmaceutical formulations comprising a therapeutically effective amount of ezogabine and one or more pharmaceutically acceptable excipients.

In certain embodiments, the term “pharmaceutically acceptable excipient” or “excipient” includes without limitation any inactive material that is combined with ezogabine as disclosed herein to produce an immediate-release oral pharmaceutical formulation for oral administration to a mammal in need thereof, preferably a human, more preferably an infant or child. The term “pharmaceutically acceptable excipient” is intended to include, but is not limited to, binders, fillers, anti-oxidants, starch, adsorbents, suspending agents, dissolution enhancers, diluents, anti-adherents, coating agents and disintegrants which have been approved by a regulatory agency, such as for example, but is not limited to, the United States Food and Drug Administration, the European Medicines Agency or Health Canada, as being acceptable for use in a formulation for the oral administration of a pharmacologically active ingredient, and/or are considered as Generally Recognized As Safe materials (GRAS materials), and/or are listed in the Inactive Ingredients Guide published by the United States Food and Drug Administration. “Pharmaceutically acceptable excipient” can also comprise the acceptable excipients listed in Remington: The Science and Practice of Pharmacy, Fox, 21st ed. 2005.

In certain embodiments, the present disclosure is directed to pharmaceutically acceptable excipients which are useful as binders in the final formulation. Exemplary binders as pharmaceutically acceptable excipients for the immediate-release oral pharmaceutical formulations disclosed herein include, but are not limited to, acacia, agar, alginic acid, calcium carbonate, calcium lactate, carbomers, carboxymethylcellulose sodium, carrageenan, cellulose acetate phthalate, ceratonia, chitosan, copovidone, cottonseed oil, dextrates, dextrin, dextrose, ethylcellulose, gelatin, glyceryl behenate, guar gum, hydrogenated vegetable oil type I, hydroxyethyl cellulose, hydroxyethylmethyl cellulose, hydroxypropyl cellulose, hydroxypropyl starch, hypromellose, inulin, lactose, liquid glucose, magnesium aluminum silicate, maltodextrin, maltose, methylcellulose, microcrystalline cellulose, pectin, poloxamer, polycarbophil, polydextrose, polyethylene oxide, polymethacrylates, povidone, sodium alginate, starch, starch pregelatinized, stearic acid, sucrose, sunflower oil, tricaprylin, vitamin E polyethylene glycol succinate and zein.

In certain embodiments, the present disclosure is directed to pharmaceutically acceptable excipients which are useful as fillers in the final formulation. Exemplary fillers as pharmaceutically acceptable excipients for the immediate-release oral pharmaceutical formulations disclosed herein include, but are not limited to, ammonium alginate, calcium carbonate, calcium lactate, calcium phosphate, calcium silicate, calcium sulfate, cellulose, cellulose—silicified microcrystalline, cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, lactose, magnesium carbonate, magnesium oxide, maltodextrin, maltose, mannitol, medium-chain triglycerides, microcrystalline cellulose, polydextrose, polymethacrylates, simethicone, sodium alginate, sodium chloride, sorbitol, starch, starch pregelatinized, starch—sterilizable maize, sucrose, sugar spheres, sulfobutylether β-cyclodextrin, talc, tragacanth, trehalose, and xylitol.

In certain embodiments, the present disclosure is directed to pharmaceutically acceptable excipients which are useful as anti-oxidants in the final formulation. Exemplary anti-oxidants as pharmaceutically acceptable excipients for the immediate-release oral pharmaceutical formulations disclosed herein include, but are not limited to, alpha tocopherol, ascorbic acid, ascorbyl palmitate, butylated hydroxyanisole, butylated hydroxytoluene, carbon dioxide, chelating agents, citric acid monohydrate, erythorbic acid, ethyl oleate, fumaric acid, malic acid, methionine, monothioglycerol, phosphoric acid, potassium metabisulfite, propionic acid, propyl gallate, sodium ascorbate, sodium bisulfite, sodium formaldehyde sulfoxylate, sodium metabisulfite, sodium sulfite, sodium thiosulfate, sulfur dioxide, tartaric acid, thymol, tocopherol, Vitamin E, and Vitamin E polyethylene glycol succinate.

In certain embodiments, the present disclosure is directed to pharmaceutically acceptable excipients which are useful as disintegrants in the final formulation. Exemplary disintegrants as pharmaceutically acceptable excipients for the immediate-release oral pharmaceutical formulations disclosed herein include, but are not limited to, alginic acid, calcium alginate, carboxymethylcellulose calcium, carboxymethylcellulose sodium, cellulose, chitosan, colloidal silicon dioxide, corn starch and pregelatinized starch, croscarmellose sodium, crospovidone, docusate sodium, glycine, guar gum, hydroxypropyl cellulose, magnesium aluminum silicate, methylcellulose, microcrystalline cellulose, polacrilin potassium, povidone, sodium alginate, sodium starch glycolate, starch, and starch pregelatinized.

In some embodiments of the invention, the present disclosure is directed to the immediate-release oral pharmaceutical formulations comprising a therapeutically effective amount of ezogabine and one or more of the following pharmaceutically acceptable excipients as described below:

In an embodiment, the present disclosure is directed to immediate-release oral pharmaceutical formulations comprising egozabine and starch as a pharmaceutically acceptable excipient, preferably wherein the starch is Starch 1500®, which is a partially pregelatinized maize starch. Starch 1500® combines several properties in a single product: binder, disintegrant, filler and flow-aid while having lubricant properties and can be used in a variety of processing methods for solid oral and dosage forms, including direct compression, wet granulation, dry granulation/roller compaction, and encapsulation.

Starch, preferably Starch 1500®, is well known as a pharmaceutically acceptable excipient for the following uses (with the typical weight (% w/w) used):

tablet and capsule diluent (up to 90%);

tablet and capsule disintegrant (3-25% (typically 15%));

tablet binder (3-20% (typically 15%)); and

anti-adherent (3-10%).

In another embodiment, the present disclosure is directed to immediate-release oral pharmaceutical formulations comprising egozabine and microcrystalline cellulose as a pharmaceutically acceptable excipient. Microcrystalline cellulose (MCC) refers to refined wood pulp and is used as a texturizer, an anti-caking agent, a fat substitute, an emulsifier, an extender, and a bulking agent in food production. The most common form is used in vitamin supplements or tablets. MCC is also used in plaque assays for counting viruses, as an alternative to carboxymethylcellulose.

Microcrystalline cellulose is well known as a pharmaceutically acceptable excipient for the following uses (with the typical weight (% w/w) used):

adsorbent (20-90%);

anti-adherent (5-20%);

capsule binder/diluent (20-90%);

tablet disintegrant (5-15%); and

tablet binder/diluent (20-90%).

In another embodiment, the present disclosure is directed to immediate-release oral pharmaceutical formulations comprising egozabine and polyvinylpyrrolidone as a pharmaceutically acceptable excipient. Polyvinylpyrrolidone (PVP), also known as polyvidone or povidone, is a water-soluble polymer made from the monomer N-vinylpyrrolidone. It is used as a binder in many pharmaceutical tablets; it simply passes through the body when taken orally.

Polyvinylpyrrolidone is well known as a pharmaceutically acceptable excipient for the following uses (with the typical weight (% w/w) used):

carrier (10-25%);

coating agent (0.5-5%);

disintegrant (5-15%)

dissolution enhancer (5%);

suspending agent (<5%); and

tablet binder (0.5-5%),

In another embodiment, the present disclosure is directed to immediate-release oral pharmaceutical formulations comprising egozabine and hydroxypropyl methylcellulose as a pharmaceutically acceptable excipient. Hydroxypropyl methylcellulose (HPMC), also known as hypromellose, is a semisynthetic, inert, viscoelastic polymer used, for example, as eye drops, as well as an excipient and controlled-delivery component in oral pharmaceutical formulations as well as in other commercial products.

Hydroxpropyl methylcelluose is well known as a pharmaceutically acceptable excipient for the following uses (with the typical weight (% w/w) used):

bioadhesive material (<5%);

coating agent (<5%);

controlled-release agent (10-80%);

dispersing agent (0.25-5%);

extended-release agent (10-80%);

film-forming agent (2-20%);

foaming agent (<1%);

granulation aid (2-5%);

mucoadhesive (0.1%);

release-modifying agent (10-80%);

solubilizing agent (<1%);

stabilizing agent (<5%);

suspending agent (<5%);

sustained-release agent (10-80%);

tablet binder (2-5%); and

viscosity-increasing agent (0.45-1%).

In one embodiment, the present disclosure is directed to immediate-release oral pharmaceutical formulations comprising egozabine and butylated hydroxytoluene as a pharmaceutically acceptable excipient. Butylated hydroxytoluene (BHT), also known as dibutylhydroxytoluene or 2,6-di-tert-butyl-4-methylphenol, is a lipophilic organic compound, chemically a derivative of phenol, that is useful for its antioxidant properties.

In one embodiment, the present disclosure is directed to immediate-release oral pharmaceutical formulations comprising egozabine and a crospovidone as a pharmaceutically acceptable excipient. Crospovidones, preferably Polyplasdone XL®, are used as disintegrants and dissolution agents for solid oral dosage forms in pharmaceuticals, and are even effective for poorly soluble dosage forms. A disintegrant is a pharmaceutically acceptable excipient used in the preparation of tablets, which causes them to disintegrate and release their active pharmaceutical ingredient on contact with moisture.

Polyplasdone XL® is well known as a pharmaceutically acceptable excipient as a tablet disintegrant in a concentration of from about 0.1% w/w to about 10% w/w, preferably from about 2% w/w to about 5% w/w.

In certain embodiments, the present disclosure is directed to immediate-release oral pharmaceutical formulations comprising a therapeutically effective amount of ezogabine and one or more pharmaceutically acceptable excipients selected from starch, microcrystalline cellulose, polyvinylpyrrolidone, hydroxypropyl methylcellulose, butylated hydroxytoluene and crospovidone.

In certain embodiments, the present disclosure is directed to immediate-release oral pharmaceutical formulations comprising a therapeutically effective amount of ezogabine and two or more pharmaceutically acceptable excipients selected from starch, microcrystalline cellulose, polyvinylpyrrolidone, hydroxypropyl methylcellulose, butylated hydroxytoluene and crospovidone.

In certain embodiments, the present disclosure is directed to immediate release oral pharmaceutical formulations comprising a therapeutically effective amount of ezogabine and three or more pharmaceutically acceptable excipients selected from starch, microcrystalline cellulose, polyvinylpyrrolidone, hydroxypropyl methylcellulose, butylated hydroxytoluene and crospovidone.

In certain embodiments, the present disclosure is directed to immediate release oral pharmaceutical formulations comprising a therapeutically effective amount of ezogabine and four or more pharmaceutically acceptable excipients selected from starch, microcrystalline cellulose, polyvinylpyrrolidone, hydroxypropyl methylcellulose, butylated hydroxytoluene and crospovidone.

In certain embodiments, the present disclosure is directed to immediate-release oral pharmaceutical formulations comprising a therapeutically effective amount of ezogabine and pharmaceutically acceptable excipients selected from starch, microcrystalline cellulose, hydroxypropyl methylcellulose, butylated hydroxytoluene and crospovidone.

In certain embodiments, the present disclosure is directed to immediate-release oral pharmaceutical formulations comprising a therapeutically effective amount of ezogabine and one or more pharmaceutically acceptable excipients, wherein each pharmaceutically acceptable excipient is present in a concentration of from about 0.01% w/w to about 99% w/w.

In certain embodiments, the present disclosure is directed to immediate-release oral pharmaceutical formulations comprising a therapeutically effective amount of ezogabine and one or more pharmaceutically acceptable excipients selected from starch, microcrystalline cellulose, hydroxypropyl methylcellulose, polyvinylpyrrolidone, butylated hydroxytoluene and/or crospovidone, wherein each pharmaceutically acceptable excipient is present in a concentration of from about 0.01% w/w to about 99% w/w.

In certain embodiments, the present disclosure is directed to immediate-release oral pharmaceutical formulations comprising a therapeutically effective amount of ezogabine and one or more pharmaceutically acceptable excipients selected from starch, microcrystalline cellulose, hydroxypropyl methylcellulose, polyvinylpyrrolidone, butylated hydroxytoluene and/or crospovidone, wherein the starch is present in a concentration of from about 5.0% w/w to about 90% w/w; the microcrystalline cellulose is present in a concentration of from about 5.0% w/w to about 90% w/w; the hydroxypropyl methylcellulose is present in a concentration of from about 0.1% w/w to about 80% w/w; the butylated hydroxytoluene is present in a concentration of from about 0.001% w/w to about 2.0% w/w; the polyvinylpyrrolidone is present in a concentration of from about 0.1% w/w to about 25% w/w; and the crospovidone is present in a concentration of from about 1.0% w/w to about 10% w/w.

In certain embodiments, the present disclosure is directed to immediate-release oral pharmaceutical formulations comprising a therapeutically effective amount of ezogabine and one or more pharmaceutically acceptable excipients selected from starch, microcrystalline cellulose, hydroxypropyl methylcellulose, butylated hydroxytoluene and crospovidone, wherein the starch is present in a concentration of from about 5.0% w/w to about 90% w/w; the microcrystalline cellulose is present in a concentration of from about 5.0% w/w to about 90% w/w; the hydroxypropyl methylcellulose is present in a concentration of from about 0.1% w/w to about 80% w/w; the butylated hydroxytoluene is present in a concentration of from about 0.001% w/w to about 2.0% w/w; and the crospovidone is present in a concentration of from about 1.0% w/w to about 10% w/w.

In some embodiments of the invention, the present disclosure is directed to immediate-release oral pharmaceutical formulations comprising a therapeutically effective amount of ezogabine and one or more pharmaceutically acceptable excipients selected from starch, microcrystalline cellulose, hydroxypropyl methylcellulose, butylated hydroxytoluene and crospovidone, wherein the starch is present in a concentration of from about 20.0% w/w to about 90% w/w; the microcrystalline cellulose is present in a concentration of from about 15.0% w/w to about 45% w/w; the hydroxypropyl methylcellulose is present in a concentration of from about 5.0% w/w to about 20% w/w; the butylated hydroxytoluene is present in a concentration of from about 0.01% w/w to about 0.10% w/w; and the crospovidone is present in a concentration of from about 1.0% w/w to about 10% w/w.

In some embodiment of the invention, the present disclosure is directed to immediate-release oral pharmaceutical formulations comprising a therapeutically effective amount of ezogabine and one or more pharmaceutically acceptable excipients selected from starch, microcrystalline cellulose, hydroxypropyl methylcellulose, butylated hydroxytoluene and crospovidone, wherein the starch is present in a concentration of about 20.0% w/w; the microcrystalline cellulose is present in a concentration of about 45% w/w; the hydroxypropyl methylcellulose is present in a concentration of about 5.0% w/w; the butylated hydroxytoluene is present in a concentration of about 0.01% w/w; and the crospovidone is present in a concentration of about 10% w/w.

### Dosing Ranges For Pharmaceutical Formulations Of The Invention

In some embodiments, the present disclosure is directed to immediate-release oral pharmaceutical formulations comprising an effective amount of ezogabine and one or more pharmaceutically acceptable excipients which are useful in treating epilepsy and/or epileptic seizure disorders in children, particularly for KCNQ2-related neonatal developmental and epileptic encephalopathy (KCNQ2-DEE), wherein the immediate-release oral pharmaceutical formulations allow for flexible weight-based dosing without requiring extemporaneous compounding of the active pharmaceutical agent.

“Weight-based dosing” refers to the practice of dosing a subject with a dose of a drug which is proportional to the subject's body weight. This is in contrast to fixed-strength dosing, where a subject is dosed in a manner which is independent of their body weight. An example of a weight-based dosing regimen would be “take 3 mg of drug per kg of body weight twice daily”, whereas an example of fixed-strength dosing would be “take 250 mg of drug twice daily”. Weight-based dosing is particularly common in a pediatric setting, as pediatric subjects (i.e., children) generally have greater ranges of body weights when compared to adults.

In some embodiments, the present disclosure is directed to immediate-release oral pharmaceutical formulations comprising a therapeutically effective amount of ezogabine and one or more pharmaceutically acceptable excipients wherein the immediate-release oral pharmaceutical formulation is orally administered to a mammal, preferably a human, more preferably a child, once a day (qd), i.e. one dose per 24 hours.

In some embodiments, the present disclosure is directed to immediate-release oral pharmaceutical formulations comprising a therapeutically effective amount of ezogabine and one or more pharmaceutically acceptable excipients wherein the immediate-release oral pharmaceutical formulation is orally administered to a mammal, preferably a human, more preferably a child, twice a day (bid), i.e., two doses per 24 hours.

In some embodiments, the present disclosure is directed to immediate-release oral pharmaceutical formulations comprising a therapeutically effective amount of ezogabine and one or more pharmaceutically acceptable excipients wherein the immediate-release oral pharmaceutical formulation is orally administered to a mammal, preferably a human, more preferably a child, three times a day (tid), i.e., three doses per 24 hours.

In some embodiments, the present disclosure is directed to immediate-release oral pharmaceutical formulations comprising a therapeutically effective amount of ezogabine and one or more pharmaceutically acceptable excipients wherein the immediate-release oral pharmaceutical formulation is orally administered to a mammal, preferably a human, more preferably a child, four times a day (qid), i.e., four doses per 24 hours.

In some embodiments, the present disclosure is directed to immediate-release oral pharmaceutical formulations comprising a therapeutically effective amount of ezogabine and one or more pharmaceutically acceptable excipients wherein the therapeutically effective amount of ezogabine is between about 1 mg/kg/dose and 7 mg/kg/dose.

In certain embodiments, a therapeutically effective amount of ezogabine in a immediate-release oral pharmaceutical formulation disclosed herein which is orally administered to a mammal, preferably a human, more preferably a child, as one dose per 24 hours, is between about 1 mg/kg/day and about 7 mg/kg/day.

In certain embodiments, a therapeutically effective amount of ezogabine in a immediate-release oral pharmaceutical formulation disclosed herein which is orally administered to a mammal, preferably a human, more preferably a child, as two doses per 24 hours is between about 2 mg/kg/day and about 14 mg/kg/day.

In certain embodiments, the therapeutically effective amount of ezogabine in a immediate-release oral pharmaceutical formulation disclosed herein which is orally administered to a mammal, preferably a human, more preferably a child, as three doses per 24 hours is between about 3 mg/kg/day and about 21 mg/kg/day.

In certain embodiments, the therapeutically effective amount of ezogabine in a immediate-release oral pharmaceutical formulation disclosed herein which is orally administered to a mammal, preferably a human, more preferably a child, as four doses per 24 hours is between about 4 mg/kg/day and about 28 mg/kg/day.

In some embodiments, the present disclosure is directed to individual immediate-release oral pharmaceutical formulations as disclosed herein comprising a therapeutically effective amount of egozabine and one or more pharmaceutically acceptable excipients, wherein the individual immediate-release oral pharmaceutical formulation provides the desired therapeutically effective amount of egozabine for the daily dose.

In some embodiments, the present disclosure is directed to immediate-release oral pharmaceutical formulations comprising a therapeutically effective amount of ezogabine and one or more pharmaceutically acceptable excipients, wherein ezogabine is present in a concentration of from about 1% w/w to about 30% w/w.

In some embodiments, the present disclosure is directed to immediate-release oral pharmaceutical formulations comprising a therapeutically effective amount of ezogabine and one or more pharmaceutically acceptable excipients, wherein ezogabine is present in a concentration of from about 5% w/w to about 20% w/w.

In some embodiments, the present disclosure is directed to immediate-release oral pharmaceutical formulations comprising a therapeutically effective amount of ezogabine and one or more pharmaceutically acceptable excipients, wherein ezogabine is present in a concentration of from about 10% w/w to about 20% w/w.

In some embodiments, the present disclosure is directed to immediate-release oral pharmaceutical formulations comprising a therapeutically effective amount of ezogabine and one or more pharmaceutically acceptable excipients, wherein ezogabine is present in a concentration of about 20% w/w.

In order to encompass all of the individual doses of the immediate-release oral pharmaceutical formulations disclosed herein, wherein ezogabine is present in a concentration of about 20% w/w, in the range of about 4 mg (a 1 mg/kg dose of the immediate-release oral pharmaceutical formulation to a 4 kg infant) to about 140 mg (1 7 mg/kg dose of the immediate-release oral pharmaceutical formulation to a 20 kg child), the following embodiments of individual doses of a immediate-release oral pharmaceutical formulation disclosed herein were prepared and filled into appropriate containers, such as HPMC sprinkle capsules or sachets:

15 mg of an immediate-release oral pharmaceutical formulation of the invention (comprising 3 mg ezogabine (20% w/w));

60 mg of an immediate-release oral pharmaceutical formulation of the invention (comprising 12 mg ezogabine (20% w/w)); and

160 mg of an immediate-release oral pharmaceutical formulation of the invention (comprising 32 mg ezogabine (20% w/w)).

These doses of the immediate-release oral pharmaceutical formulations of the invention, when taken individually (such as a 15 mg dose of a immediate-release oral pharmaceutical formulation of the invention comprising 3 mg of ezogabine once, twice, three times or four times a day) or in combination (such as a 15 mg dose of a immediate-release oral pharmaceutical formulation of the invention as the first dose in a day and a 60 mg of a immediate-release oral pharmaceutical formulation of the invention as the second dose in a day and so forth) would be effective in providing a therapeutically effective amount of egozabine in the range of 1 mg/kg/dose and 7 mg/kg/dose. For example, and not intended to limit the scope of this disclosure, if a 10 kg child is to be dosed with a therapeutically effective amount of ezogabine at 3 mg/kg/day in three doses, the child would receive 30 mg of ezogabine per 24 hours (at 10 mg/kg/dose). If each dose of a immediate-release oral pharmaceutical formulation of the invention contained 20% w/w of egozabine, the child would need to receive 150 mg of the immediate-release oral pharmaceutical formulation of the invention each day (each dose would be 50 mg of the immediate-release oral pharmaceutical formulation) in order to receive 3 mg/kg/day of ezogabine.

In some embodiments, this disclosure is directed to the oral administration of individual doses of an immediate-release oral pharmaceutical formulation of the invention to the child, by emptying the requisite number of individual doses into infant/children food, such as, but not limited to, breast milk, infant formula, cow's milk, soy milk, almond milk, nut milk, fruit juice or soft food (e.g., apple sauce, pudding, yogurt, pureed foods), stirring well and orally administering the infant food to the child in need thereof.

### Utility of the Pharmaceutical Formulations of the Invention

In some embodiments, the present disclosure is directed to immediate-release oral pharmaceutical formulations comprising an effective amount of ezogabine and one or more pharmaceutically acceptable excipients which are useful in treating epilepsy and/or epileptic seizure disorders in a mammal, preferably a human, more preferably a child, particularly wherein the epilepsy and/or epileptic seizure disorder is KCNQ2-related neonatal developmental and epileptic encephalopathy (KCNQ2-DEE).

There is strong human genetic validation and pharmacologic evidence, including published case studies that support the use of the immediate-release oral pharmaceutical formulations disclosed herein as a potential treatment for KCNQ2-DEE. The KCNQ2 gene encodes for the Kv7.2 voltage-gated potassium channel. Loss-of-function missense mutations in KCNQ2 can cause KCNQ2-DEE, which is characterized in general, by multiple, daily, treatment-resistant seizures often presenting within the first week of life. The immediate-release oral pharmaceutical formulations disclosed herein may have a greater potential to improve long term outcomes in the treatment of KCNQ2-DEE, as ezogabine enhances transmembrane potassium currents mediated by the Kv7.2/7.3 channels, thus potentially reversing the underlying genetic abnormality of KCNQ2-DEE. By activating Kv7.2/7.3 channels, it is expected that the immediate-release oral pharmaceutical formulations disclosed herein should stabilize the resting membrane potential and reduce brain excitability and may have the potential to improve brain function and cognitive development, in addition to decreasing seizures. In one previously published case report of 11 patients (Millichap, J. J. et al., Neurol. Genet., October 2016, 2:1-5), ezogabine was associated with improvement in seizures and/or development in three of the four infants treated before six months of age, and two of the seven treated later. No serious adverse effects were observed in that study. Another study that included a review of medical records and structured interviews with families of eight children with KCNQ2-DEE who had previously been prescribed ezogabine (Olson, H. et al., Annual Meeting of the American Epilepsy Society 2017: Abstract 3.176), also suggested that ezogabine was effective and tolerable. Sustained improvement in seizure frequency was observed in five of the six patients with at least weekly seizures, along with improvements in development or cognition in all eight patients.

In other embodiments, the immediate-release oral pharmaceutical formulations disclosed herein are useful in treating KCNQ2-related neonatal developmental and epileptic encephalopathy (KCNQ2-DEE), partial seizures (such as simple, complex, secondary generalized, and focal onset), generalized seizures (such as absence, myoclonic, atonic, tonic and tonic clonic), and disorders including photosensitive epilepsy, self-induced syncope, intractable epilepsy, Angelman syndrome, benign rolandic epilepsy, CDKL5 disorder, childhood and juvenile absence epilepsy, Dravet syndrome, frontal lobe epilepsy, Glut1 deficiency syndrome, hypothalamic hamartoma, infantile spasms/West's syndrome, juvenile myoclonic epilepsy, Landau-Kleffner syndrome, Lennox-Gastaut syndrome (LGS), epilepsy with myoclonic-absences, Ohtahara syndrome, Panayiotopoulos syndrome, PCDH19 epilepsy, progressive myoclonic epilepsies, Rasmussen's syndrome, ring chromosome 20 syndrome, reflex epilepsies, temporal lobe epilepsy, Lafora progressive myoclonus epilepsy, neurocutaneous syndromes, tuberous sclerosis complex, early infantile epileptic encephalopathy, early onset epileptic encephalopathy, generalized epilepsy with febrile seizures plus (GEFS+), Rett syndrome, multiple sclerosis, Alzheimer's disease, autism, ataxia, hypotonia and paroxysmal dyskinesia.

In some embodiments, the present disclosure is directed to methods of treating epilepsy and/or an epileptic seizure disorder in a mammal, particularly KCNQ2-related neonatal developmental and epileptic encephalopathy (KCNQ2-DEE), wherein the method comprises orally administering to the mammal in need thereof an immediate-release oral pharmaceutical formulation as disclosed herein comprising a therapeutically effective amount of ezogabine and one or more pharmaceutically acceptable excipients.

In some embodiments, the present disclosure is directed to methods of treating epilepsy and/or an epileptic seizure disorder in a human, particularly KCNQ2-related neonatal developmental and epileptic encephalopathy (KCNQ2-DEE), wherein the method comprises orally administering to the mammal in need thereof an immediate-release oral pharmaceutical formulation as disclosed herein comprising a therapeutically effective amount of ezogabine and one or more pharmaceutically acceptable excipients.

In some embodiments, the present disclosure is directed to methods of treating epilepsy and/or an epileptic seizure disorder in a child, wherein the method comprises orally administering to the mammal in need thereof an immediate-release oral pharmaceutical formulation as disclosed herein comprising a therapeutically effective amount of ezogabine and one or more pharmaceutically acceptable excipients.

In some embodiments, the present disclosure is directed to methods of treating epilepsy and/or an epileptic seizure disorder in a child, wherein the method comprises orally administering to the mammal in need thereof an immediate-release oral pharmaceutical formulation as disclosed herein comprising a therapeutically effective amount of ezogabine and one or more pharmaceutically acceptable excipients, wherein the immediate-release oral pharmaceutical formulation comprises two or more pharmaceutically acceptable excipients.

In some embodiments, the present disclosure is directed to methods of treating epilepsy and/or an epileptic seizure disorder in a child, wherein the method comprises orally administering to the mammal in need thereof an immediate-release oral pharmaceutical formulation as disclosed herein comprising a therapeutically effective amount of ezogabine and one or more pharmaceutically acceptable excipients, wherein the immediate-release oral pharmaceutical formulation comprises two or more pharmaceutically acceptable excipients, wherein each pharmaceutically acceptable excipient is present in a concentration of from about 0.01% w/w to about 99% w/w.

In some embodiments, the present disclosure is directed to methods of treating epilepsy and/or an epileptic seizure disorder in a mammal, particularly KCNQ2-related neonatal developmental and epileptic encephalopathy (KCNQ2-DEE), wherein the method comprises orally administering to the mammal in need thereof an immediate-release oral pharmaceutical formulation as disclosed herein comprising a therapeutically effective amount of ezogabine and one or more pharmaceutically acceptable excipients, wherein ezogabine is present in a concentration of from about 1% w/w to about 30% w/w.

In some embodiments, the present disclosure is directed to methods of treating epilepsy and/or an epileptic seizure disorder in a mammal, particularly KCNQ2-related neonatal developmental and epileptic encephalopathy (KCNQ2-DEE), wherein the method comprises orally administering to the mammal in need thereof an immediate-release oral pharmaceutical formulation as disclosed herein comprising a therapeutically effective amount of ezogabine and one or more pharmaceutically acceptable excipients, wherein the immediate release pharmaceutical composition comprises ezogabine at a concentration of about 20% w/w; HPMC at a concentration of about 5.0% w/w; starch at a concentration of about 20% w/w; MCC at a concentration of about 45% w/w; butylated hydroxytoluene at a concentration of about 0.01% w/w; and crosprovidone at a concentration of about 10.0% w/w of the immediate-release oral pharmaceutical formulation.

### Preparation of Pharmaceutical Formulations of the Invention and Stability and Dissolution Studies Thereof

The development and preparation of the immediate-release oral pharmaceutical formulations disclosed herein utilized a modified quality-by-design approach. Ezogabine exhibits low water solubility and high permeability and is therefore classified in the Biopharmaceutics Classification System as a Class 2 compound. Accordingly, excipient compatibility for ezogabine was determined through an accelerated-condition (40° C., 75% relative humidity) stability study of different formulations of ezogabine and various excipients. In vitro dissolution profiles of the lead formulations were also determined.

Formulations with the most promising dissolution profiles were then dry granulated through roller compaction and re-tested for dissolution prior to stability assessments.

The impact of ezogabine loading on dissolution performance was also determined, along with the potential for non-specific binding of the immediate-release oral pharmaceutical formulations of the invention to common plastics such as those employed in feeding bottles and nasogastric feeding tubes.

The preferred immediate-release oral pharmaceutical formulation of the invention was then advanced to rat pharmacokinetic (PK) studies in order to confirm its biopharmaceutical performance in vivo and placed on long-term stability studies.

It is understood that that one skilled in the art would be able to prepare the immediate-release oral pharmaceutical formulations disclosed herein, as specifically illustrated below as Examples 1-20, by methods known to one skilled in the art. It is also understood that one skilled in the art would be able to prepare in a similar manner other immediate-release oral pharmaceutical formulations of the invention not specifically illustrated below by using the appropriate components and modifying the parameters of the preparation as needed.

Accordingly, in some embodiments, the present disclosure is directed to methods of preparing an immediate-release oral pharmaceutical formulation as disclosed herein comprising a therapeutically effective amount of ezogabine and one or more pharmaceutically acceptable excipients.

In some embodiments, the present disclosure is directed to methods of preparing an immediate-release oral pharmaceutical formulation as disclosed herein comprising a therapeutically effective amount of ezogabine and two or more pharmaceutically acceptable excipients.

In some embodiments, the present disclosure is directed to methods of preparing an immediate-release oral pharmaceutical formulation as disclosed herein comprising a therapeutically effective amount of ezogabine and one or more pharmaceutically acceptable excipients, wherein each pharmaceutically acceptable excipient is present in a concentration of from about 0.01% w/w to about 99% w/w.

In some embodiments, the present disclosure is directed to methods of preparing an immediate-release oral formulation formulation as disclosed herein comprising a therapeutically effective amount of ezogabine and one or more pharmaceutically acceptable excipients, wherein ezogabine is present in a concentration of from about 1% w/w to about 30% w/w.

In some embodiments, the present disclosure is directed to methods of preparing an immediate-release oral pharmaceutical formulation as disclosed herein comprising a therapeutically effective amount of ezogabine and one or more pharmaceutically acceptable excipients, wherein the immediate-release oral pharmaceutical formulation is as disclosed herein in Example 1, Example 2, Example 3, Example 4, Example 5, Example 6, Example 7, Example 8, Example 9, Example 10, Example 11, Example 12, Example 13, Example 14, Example 15, Example 16, Example 17, Example 18, Example 19, or Example 20.

In some embodiments, the present disclosure is directed to methods of preparing an immediate-release oral pharmaceutical formulation as disclosed herein comprising a therapeutically effective amount of ezogabine and one or more pharmaceutically acceptable excipients, wherein the immediate-release formulation comprises ezogabine at a concentration of about 20% w/w; HPMC at a concentration of about 5.0% w/w; starch at a concentration of about 20% w/w; MCC at a concentration of about 45% w/w; butylated hydroxytoluene at a concentration of about 0.01% w/w; and crosprovidone at a concentration of about 10.0% w/w of the immediate-release oral pharmaceutical formulation.

In some embodiments, the present disclosure is directed to methods of preparing an immediate-release oral pharmaceutical formulation as disclosed herein wherein the preparation comprises mixing the ingredients together, followed by granulation.

In some embodiments, the granulated immediate-release oral pharmaceutical formulations prepared herein are enclosed within a container which can be in the form of an ampoule, capsule, sachet, paper, or other container. In some embodiments, the containers for the granulated formulations disclosed herein are capsules (gelatin or HPMC), sprinkle capsules (gelatin or HPMC), sachets, stick packs or pre-filled oral syringes (in which the granules can be suspended in a suitable vehicle). In some embodiments, the containers for the granulated formulations disclosed herein are vials, ampoules or blister packages.

In some embodiments, the granulated immediate-release oral pharmaceutical formulations disclosed herein are enclosed within a sachet. Sachets are typically foil or plastic pouches which are filled with the granulated immediate-release oral pharmaceutical formulations disclosed herein and then sealed. The sachets preferably contain a single dose.

In some embodiments, the granulated immediate-release oral pharmaceutical formulations disclosed herein are enclosed in a sprinkle capsule. Sprinkle capsules resemble standard 2-part gelatin capsules or HPMC capsules, except that they are designed to be easier to open (separating the cap from the base is done with a quarter-turn twist, and the two parts separate easily). In contrast, standard 2-part gelatin capsules or HPMC capsules have to be pulled apart, which could lead to loss of the granulated formulation contained therein through spillage.

### Experimental Procedure for Stability Study of Representative Pharmaceutical Formulations of the Invention

Approximately 200 mg of each representative immediate-release oral pharmaceutical formulation of the invention was prepared by thorough mixing of the active pharmaceutical ingredient (ezogabine) and the relevant pharmaceutically acceptable excipient, as indicated below in Table 2. The representative formulations of Examples 1-8 were analyzed both prior to storage, as well as following 4 weeks of storage in open glass vials in a stability chamber set to maintain 40±2° C. and 75±5% relative humidity. Stability analysis was performed on the representative formulations using the following method and the sum of total related substances (i.e., degradants) was determined as set forth below in Table 1 where “ACN” is acetonitrile:

TABLE 1

| Instrument | Shimadzu | | |
|---|---|---|---|
| Column | Halo ® C18, 150 × 4.6 mm, 2.7 μm | | |
| Mobile phase | Mobile Phase A: 0.1% H₃PO₄ in water v/v | | |
| | Mobile Phase B: ACN | | |
| Gradient | Time (min) | A (%) | B (%) |
| | 0 | 95 | 5 |
| | 18 | 5 | 95 |
| | 23 | 5 | 95 |
| | 28 | 95 | 5 |

| | |
|---|---|
| Run Time | 28 minutes |
| Column Temp | 30° C. |
| Auto Sampler Temp | Room temperature |
| Flow rate | 1.0 mL/min |
| Injection volume | 10 μL with needle wash |
| Wavelength | UV 254 nm |
| | Bandwidth 4 nm, Reference Wavelength Off |
| Diluent | ACN/water (80/20, v/v) |

In some embodiments, as set forth below as Examples 1-8, the microcrystalline cellulose (MCC)/starch system in the immediate-release oral pharmaceutical formulations of the invention, combined with the use of HPMC as a binder appeared to be the most compatible with ezogabine in producing a stable immediate-release formulation, as shown below in Table 2, wherein PVP is poly(vinylpyrrolidone), HPMC is (hydroxypropyl)methylcellulose, and BHT is butylated hydroxytoluene:

TABLE 2

| | Composition (% w/w) | | | | | | % |
|---|---|---|---|---|---|---|---|
| Example | | | Starch | | | | Increase |
| # | API | MCC | 1500 | PVP | HPMC | BHT | in TRS* |
| 1 | 5 | 45 | 44.99 | — | 5 | 0.01 | 0.38 |
| 2 | 5 | 45 | 44.99 | 5 | — | 0.01 | 0.50 |
| 3 | 5 | 42.5 | 42.49 | — | 10 | 0.01 | 0.27 |
| 4 | 5 | 37.5 | 37.49 | — | 20 | 0.01 | <0.05 |
| 5 | 5 | — | 89.99 | — | 5 | 0.01 | 0.17 |
| 6 | 5 | 89.99 | — | — | 5 | 0.01 | 0.33 |
| 7 | 5 | — | 89.99 | 5 | — | 0.01 | 0.17 |
| 8 | 5 | 17.5 | 72.49 | — | 5 | 0.01 | 0.34 |

*Percentage increase in Total Related Substances (TRS) (i.e., degradants) by HPLC/UV after 4 weeks of open storage at 40° C./75% relative humidity.

### Experimental Procedure for Dissolution Assessment of Representative Pharmaceutical Formulations of the Invention

The dissolution assessment of representative immediate-release oral pharmaceutical formulations of the invention, i.e., Examples 9-20 as shown below in Table 5, was made using the parameters set forth in Table 3 below:

TABLE 3

| | |
|---|---|
| Agitation | USP Apparatus 2 (paddle) |
| Rotation speed | 50 rpm |
| Infinity spin | 200 rpm |
| Dissolution medium | 0.1N HCl, pH 1 |
| Dissolution volume | 900 mL |
| Medium temperature | 37.0 ± 0.5° C. |
| Sample method | Auto sampler |
| Sampling volume | 5 mL |
| Sampling time | 5, 10, 15, 30 45, 60 min and infinity |
| | spin at 200 rpm for 15 min |
| Clarification | Filter disk, UHMW-PE, 10 Micron |
| Secondary filter | ANPEL PP, 0.45 μm × 25 mm |
| Sample stability | Solution stable for 67 hours at 5° C. |

The percentage of ezogabine released at each timepoint was determined by HPLC using the following method as shown below in Table 4:

TABLE 4

| Instrument | Shimadzu | | |
|---|---|---|---|
| Column | Halo ® C18, 150 × 4.6 mm, 2.7 μm | | |
| Mobile phase | Mobile Phase A: 0.1% Formic acid in water v/v | | |
| | Mobile Phase B: 0.1% Formic acid in ACN v/v | | |
| Gradient | Time (min) | A (%) | B (%) |
| | 0 | 95 | 5 |
| | 18 | 5 | 95 |
| | 23 | 5 | 95 |
| | 28 | 95 | 5 |

| | |
|---|---|
| Run Time | 28 minutes |
| Column Temp | 37° C. |
| Auto Sampler Temp | 37° C. |
| Flow rate | 1.0 mL/min |
| Injection volume | 10 μL with needle wash |
| Wavelength | UV 254 nm |
| | Bandwidth 4 nm, Reference Wavelength Off |
| Diluent | ACN/water (80/20, v/v) |

In some embodiments, superior release of egozabine was found with adding a disintegrant to the immediate-release oral pharmaceutical formulations of the invention comprising egozabine in a therapeutically effective amount of 20% w/w, as shown below in Table 5 (where “Ex.” refers to Example):

TABLE 5

| | Composition (% w/w) | | | | | | % Released |
|---|---|---|---|---|---|---|---|
| | | | | | | | at T = |
| Ex. | | | | Starch | | Other | 30/60 |
| # | API | BHT | HPMC | 1500 | MCC | Excipients | minutes* |
| 9 | 5 | 0.01 | 5 | 44.99 | 45 | — | 64.1 / 70.2 |
| 10 | 5 | 0.01 | 20 | 37.49 | 37.5 | — | 85.8 / 89.4 |
| 11 | 5 | 0.01 | 5 | 42.49 | 42.5 | Croscarmellose | 89.7 / 93.1 |
| | | | | | | sodium (5%) | |
| 12 | 20 | 0.01 | 5 | 19.99 | 45 | Polyplasdone | 96.9 / 100.5 |
| | | | | | | XL ® (10%) | |
| 13 | 5 | 0.01 | 5 | 24.99 | 25 | Lactose (40%) | 70.4 / 74.7 |
| 14 | 5 | 0.01 | 5 | 59.99 | 30 | — | 63.9 / 69.4 |
| 15 | 6.98 | 0.01 | 6.98 | 79.05 | — | Croscarmellose | 80.6 / 84.2 |
| | | | | | | sodium (6.98%) | |
| 16 | 10 | 0.01 | 5 | 29.99 | 45 | Polyplasdone | 80.3 / 82.1 |
| | | | | | | XL ® (10%) | |
| 17 | 10 | 0.01 | 5 | 29.99 | 45 | Polyplasdone | 84.3 / 90.4 |
| | | | | | | Ultra ® (10%) | |
| 18 | 10 | 0.01 | 5 | 42.49 | 45 | — | 80.5 / 81.6 |
| 19 | 20 | 0.01 | 5 | 37.49 | 37.5 | — | 88.3 / 91.9 |
| 20 | 10 | 0.01 | 5 | 19.99 | 40 | Lactose (20%) + | 85.9 / 86.3 |
| | | | | | | CCN (5%) | |

*pH 1 (0.1 N HCL), 37° C., n = 6

As demonstrated in the results above, incorporation of a disintegrant, e.g., polyplasdone XL, into the immediate release oral pharmaceutical formulations of the invention led to significant improvements in the dissolution profile (see Example #12 vs. Example #9). Furthermore, superior results were obtained using a 20% w/w concentration of ezogabine (see Example #12 vs. Example #16).

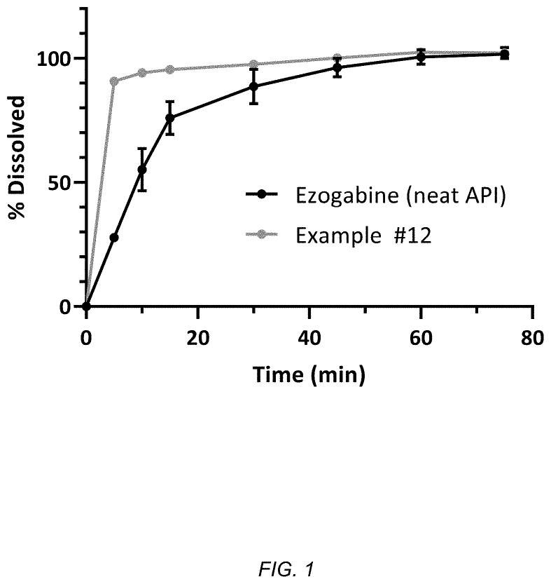

In one embodiment, Example #12 above was granulated (dry granulation via roller compaction) to afford an immediate-release oral pharmaceutical formulation of the invention. Following granulation, the dissolution profiles of ezogabine (neat) and of the granulated immediate-release oral pharmaceutical formulation were determined in USP pH 1.2 buffer at 37° C. As shown in FIG. 1, the dissolution profile of the immediate-release formulation was consistent with an immediate-release drug product.

In one embodiment, the immediate-release oral pharmaceutical formulations of the invention will be stable for a time period of between about 1 month and about 5 years when kept at a temperature between about 5° C. and about 50° C. More preferably, the immediate-release oral pharmaceutical formulations of this invention will be stable for a time period of between about 6 months and about 4 years when kept at a temperature between about 15° C. and about 45° C. Even more preferably, the immediate-release oral pharmaceutical formulations of this invention will be stable for a time period of between about 6 months and about 3 years when kept at a temperature between about 25° C. and about 40° C. In a more preferred embodiment, the immediate-release oral pharmaceutical formulations are stable when kept at a temperature of between about 25° C. and about 40° C. over a period of time such as a year, and preferably 2 years. More preferably, the immediate-release oral pharmaceutical formulations are stable for 3 years.

### Pediatric Dosing Compatibility Studies of Pharmaceutical Formulations of the Invention

In one embodiment, the present disclosure is directed to immediate-release oral pharmaceutical formulations comprising an effective amount of ezogabine and one or more pharmaceutically acceptable excipients which are useful in treating epilepsy and/or epileptic seizure disorders in children. It is therefore desirable that the immediate-release oral pharmaceutical formulations of the invention are compatible with the physical parameters of pediatric dosing and have negligible non-specific binding of the active pharmaceutical ingredient to common materials employed in baby bottles and pediatric naso-gastric feeding tubes. Accordingly, the following studies were performed on a representative immediate-release oral pharmaceutical formulation of the invention.

### Particle Size Distribution of a Representative Pharmaceutical Formulation of the Invention

In order for the immediate-release oral pharmaceutical formulations of the invention to be compatible for oral administration to a child, the particle size distribution of the immediate-release oral pharmaceutical formulations must be generally smaller in diameter than the diameter of the aperture of a typical baby bottle nipple (i.e., >500 μm) and smaller in diameter than the internal diameter of a typical pediatric naso-gastric (NG) feeding tube (e.g., 1330 μm in a Size 4 French (Fr) NG pediatric feeding tube).

The particle size distribution of the representative immediate-release oral pharmaceutical formulation of the invention, i.e., Example #12, was therefore determined by laser light scattering using a Mastersizer 3000 (Malvern Panalytical Ltd., Westborough, Mass., USA). A background measurement time of 10 seconds, a sample measurement time of 30 seconds, an air pressure of 0.5 barg and a 60% feed rate were employed in the study. The results of this study are disclosed below in Table 6:

TABLE 6

| Particle size distribution of | Size |
|---|---|
| Example #12 | (microns) |
| d₁₀ | 13.4 ± 0.4 |
| d₅₀ | 41.6 ± 1.6 |
| d₉₀ | 153 ± 31 |

As the results demonstrated, 10% of the particles in the Example #12 sample were smaller than 13 microns; 50% of the particles in the same sample were smaller than 42 microns (i.e., the median particle size); and 90% of the particles in the same sample were smaller than 153 microns. Accordingly, the particles of Example #12 are generally small enough to pass through the aperture of a typical baby bottle nipple or through a pediatric naso-gastric feeding tube.

### Non-specific Binding of a Representative Pharmaceutical Formulation of the Invention

In order to assess the non-specific binding of a representative immediate-release oral pharmaceutical formulation of the invention, the following assessment was performed.

A representative immediate-release pharmaceutical composition of the invention, i.e., Example #12, was diluted with purified water (1000 mL), sonicated for 15 minutes and filtered through a 0.45 μm filter. The filtrate was transferred into bottles made of the relevant materials (glass, polyether sulfone, polyphenyl sulfone or polypropylene) and hand-shaken for 1 minute. Aliquots of 1 mL were then withdrawn and the concentration analyzed (against a calibration curve) using the following HPLC method in Table 7 below:

TABLE 7

| Instrument | Shimadzu | | |
|---|---|---|---|
| Column | Halo ® C18, 150 × 4.6 mm, 2.7 μm | | |
| Mobile phase | Mobile Phase A: 0.1% H₃PO₄ in water v/v | | |
| | Mobile Phase B: ACN | | |
| Gradient | Time (min) | A (%) | B (%) |
| | 0 | 95 | 5 |
| | 18 | 5 | 95 |
| | 23 | 5 | 95 |
| | 28 | 95 | 5 |

| | |
|---|---|
| Run Time | 28 minutes |
| Column Temp | 30° C. |
| Auto Sampler Temp | Room temperature |
| Flow rate | 1.0 mL/min |
| Injection volume | 10 μL with needle wash |
| Wavelength | UV 254 nm |
| | Bandwidth 4 nm, Reference Wavelength Off |
| Diluent | ACN/water (80/20, v/v) |

The above assessment demonstrated only negligible non-specific binding of the active pharmaceutical ingredient, i.e., ezogabine, to common materials employed in baby bottles and pediatric naso-gastric feeding tubes, as shown below in Table 8:

TABLE 8

| Material | Recovery of API (%) |
|---|---|
| Glass | 100.0 |
| Polyether sulfone | 96.9 |
| Polyphenyl sulfone | 96.7 |
| Polypropylene | 96.5 |

### Pharmacokinetic Comparison of a Representative Immediate-Release Oral Pharmaceutical Formulation of the Invention and Potiga®.

Potiga®, a tablet formulation of ezogabine, was indicated and marketed for use in the adult population. However, pediatric neurologists were using Potiga® outside of its approved indication in the pediatric population during the time it was available (see, for example, Millichap et al.). It is appreciated that children, particularly young children, are generally unable to swallow adult solid oral dosage forms such as tablets or capsules. Typically, Potiga® would thus be taken to a compounding pharmacy, where it would be crushed (say, in a mortar and pestle) and suspended in a suitable liquid vehicle at a fixed concentration (e.g., 5 mg/mL). This compounded form of ezogabine would then be administered to pediatric patients on a volumetric basis (e.g., for a 50 mg dose, one would administer 10 mL of a 5 mg/mL suspension). As such, a rat cross-over pharmacokinetic study, as described below, was designed to incorporate a dosing group which was administered a formulation similar to suspended, crushed Potiga®.

Test articles:

Immediate-release formulation of Example #12.

Crushed Potiga® tablets (to mimic the compounding of Potiga® in previous pediatric clinical practice).

Test animals:

Male Sprague-Dawley rats, N=6

### Dosing

10 mg/kg of the test articles were suspended (1 mg/mL) in 0.02% w/v aq. (carboxymethyl)cellulose (CMC) (viscosity-matched to infant formula) and the test animals were dosed by oral gavage.

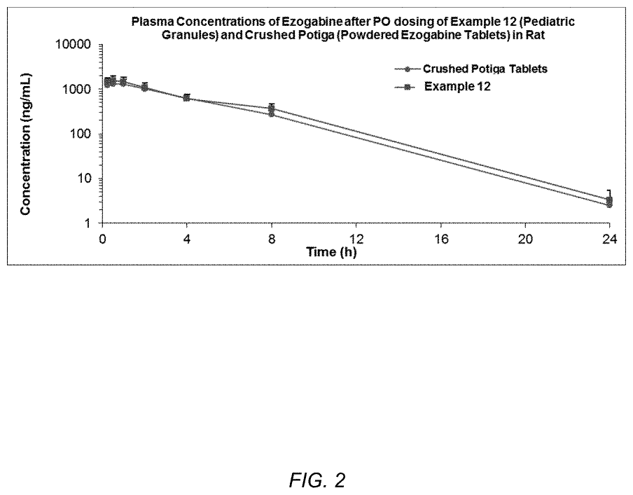

The results of this pharmacokinetic study are set forth in FIG. 2 having the following bioavailability parameters wherein “Cₘₐₓ” refers to the observed maximal plasma concentration, “AUC” refers to the area under the plasma concentration versus time curve, “AUC₀₋ₗₐₛₜ” refers to the AUC from time zero to last detectable plasma concentration and “AUC_{0-inf}” refers to the AUC from time zero to infinity:

| Parameter | Cₘₐₓ | AUC₀₋ₗₐₛₜ | AUC_{0-inf} |
|---|---|---|---|
| Lower bound of the 95% | 105.4 | 94.9 | 94.9 |
| confidence interval | | | |
| Point estimate of the geometric | 115.1 | 108.0 | 108.0 |
| mean ratio | | | |
| Upper bound of the 95% | 125.7 | 122.9 | 122.9 |
| confidence interval | | | |

The results demonstrate that a representative immediate-release oral pharmaceutical formulation of the invention exhibits a bioavailability similar to compounded Potiga® when orally administered to rats.

### In Vivo Pharmacokinetic Study of Pharmaceutical Formulations of the Invention

As described in more detail below, in another embodiment of the invention, a Phase 1 clinical study was conducted to determine the pharmacokinetic profile of ezogabine in immediate-release oral pharmaceutical formulations of the invention. In particular, healthy adult volunteers were given a single dose (2.0 grams) of an immediate-release oral pharmaceutical formulation of the invention comprising 400 mg ezogabine in either a fed or a fasted state and the plasma pharmacokinetics of ezogabine were determined. After a 7 day wash-out period, the same volunteers were again given a single dose (2.0 grams) of an immediate-release oral pharmaceutical formulation of the invention comprising 400 mg ezogabine; however, those volunteers who previously received the immediate-release oral pharmaceutical formulation in a fed state now receive it in the fasted state, and vice versa. The plasma pharmacokinetics of ezogabine were again determined. The 2.0 gram dose of the immediate-release oral pharmaceutical formulation of the invention was packaged in single-use sachets for this study.

### Protocol for the Phase 1 Clinical Study

The Phase 1, single center, open-label, randomized, single dose, 2-way crossover clinical study was conducted to evaluate the impact of food on the pharmacokinetics (PK) of ezogabine following oral administration of an immediate-release formulation of the invention, specifically the immediate-release formulation of Example #12. Safety and tolerability of the formulation were also assessed.

The study was designed to include a total of approximately 24 healthy male and female subjects. Subjects who met all inclusion criteria and none of the exclusion criteria were eligible to enter the study.

Each subject received both a single dose of the immediate release formulation of Example #12 under fasted conditions (hereinafter referred to as “Treatment A”), which was considered the reference treatment, and a single dose of the immediate release formulation of Example #12 under fed conditions (hereinafter referred to as “Treatment B”), which was considered the test treatment. Subjects were randomized equally to 1 of the 2 treatment sequences: Treatment Sequence 1, which was Treatment A followed by Treatment B, or Treatment Sequence 2, which was Treatment B followed by Treatment A, with 12 subjects per treatment sequence.

Following the screening visit, eligible subjects returned to the clinic for 2 in-clinic treatment periods (Day -1 and Day 6), each of which were separated by a 7-day wash-out period between doses.

For each subject the study consisted of:

1. An eligibility screening period of up to 28 days.
2. Two treatment periods with each involving administration of Treatment A or Treatment B.
3. Safety assessments, blood, and saliva sampling for PK purposes from predose up to 48 hours after study drug administration in each period.
4. Discharge following collection of the 48-hour PK sample in each period.
5. A follow-up visit 7 days (+1-3 days) after discharge from Period 2.

For each study period, subjects received 1 of the 2 following treatments:

Treatment A: Single dose of 400 mg ezogabine (as one 2.0 g sachet of the formulation of Example #12) under fasted conditions.
Treatment B: Single dose of 400 mg ezogabine (as one 2.0 g sachet of the formulation of Example #12) under fed conditions.
The single dose of 400 mg ezogabine was orally administered by opening the 2.0 g sachet and dispersing the contents in 8 oz. of water, which was then consumed by the subject, either in the fed state (just after breakfast) or in the fasted state (2 h before breakfast).

A total of 24 subjects were randomized to 1 of 2 treatment sequences (Sequence 1 or Sequence 2), as shown below where N is the number of subjects and PK is pharmokinetic:

Venous blood samples for PK assessments for ezogabine were collected from the subjects prior to and at 0.25, 0.5, 1, 1.5, 2, 3, 4, 8, 12, 24, and 48 hours following each administration of the formulation of Example #12. Plasma bioanalysis for ezogabine was performed using a validated liquid chromatography/tandem mass spectrometry (LC/MS-MS) method.

The PK data set included 21 evaluable subjects.

### Results of the Phase 1 Clinical Study

The results of the Phase 1 Clinical Study are shown below in Table 9 wherein h is hour, L is litre, F is bioavailability (systemic availability of the administered dose), CV is the coefficient of variation, N is the number of subjects, SD is the standard deviation, Cₘₐₓ is the maximum observed concentration, Tₘₐₓ is the time of maximum observed concentration if it occurs at more than one time point, then Tₘₐₓ is defined as the first time point with this value), AUC₀₋₁ is the area under the concentration-time curve from time zero to the time of last observed quantifiable concentration, AUC_{0-inf} is the area under the concentration-time curve extrapolated to infinity, T½ is the terminal elimination half-life, V₂/F is the apparent volume of distribution during the terminal elimination phase, and CL/F is the apparent total plasma clearance, calculated as dose/AUC_{0-inf}.

TABLE 9

| | Ezogabine | | | |
|---|---|---|---|---|
| | Formulation of | | Formulation of | |
| | Example #12, | | Example #12, | |
| | 400 mg | | 400 mg | |
| Pharmacokinetic | ezogabine, Fed | | ezogabine, Fasted | |
| Parameter | (N = 21) | | (N = 21) | |
| Cₘₐₓ (ng/mL) | | | | |
| Mean (SD) | 668 | (169) | 977 | (375) |
| CV (%) | 25.2 | | 38.4 | |
| Tₘₐₓ (h) | | | | |
| Median (Min-Max) | 3.00 | (0.50-8.00) | 2.00 | (0.50-3.05) |
| AUC₀₋ₜ (ng * h/mL) | | | | |
| Mean (SD) | 6390 | (1160) | 7070 | (1620) |
| CV (%) | 18.2 | | 23.0 | |
| AUC_{0-inf} (ng * h/mL) | | | | |
| Mean (SD) | 6460 | (1190) | 7200 | (1670) |
| CV (%) | 18.4 | | 23.2 | |
| T_{1/2} (h) | | | | |
| Mean (SD) | 7.17 | (1.06) | 8.83 | (2.04) |
| CV (%) | 14.8 | | 23.1 | |
| V_{z}/F (L) | | | | |
| Mean (SD) | 658 | (141) | 754 | (286) |
| CV (%) | 21.4 | | 37.9 | |
| CL/F (L/h) | | | | |
| Mean (SD) | 63.9 | (11.4) | 58.3 | (13.0) |

| | | |
|---|---|---|
| CV (%) | 17.8 | 22.3 |

### Pharmacokinetic Conclusions

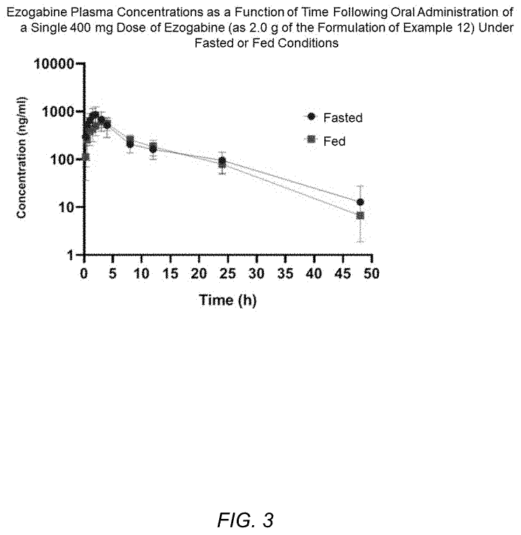

Administration of a single 400 mg dose of ezogabine (as 2.0 g of the formulation of Example #12) under fed conditions (i.e., with a high-fat meal having approximately 50 percent of the total caloric content of the meal coming from fat) slightly reduced and delayed ezogabine peak plasma concentration but did not significantly affect the extent of systemic exposure compared to the fasted state. Thus, although an absence of a food effect was not fully confirmed as the administration of food showed statistically significant effects on ezogabine's rate of absorption following single dose administration of the formulation of Example #12, systemic exposure of ezogabine was not significantly affected if the immediate-release formulation of the invention, specifically the immediate-release formulation of Example #12 was orally administered in either a fasted or a fed state, as shown in FIG. 3.

In addition, the results indicated that, following the oral administration of a 400 mg single-dose of ezogabine (as 2.0 g of the formulation of Example #12), the median Tₘₐₓ of ezogabine was delayed by 1 hour when administered with a high-fat meal versus when administered in the fasted state. However, the ezogabine in the formulation of Example #12 was sufficiently bioavailable when orally administered in either state (ezogabine was widely distributed within the body with V₂/F of 658 L and 754 L under fed and fasted states, respectively, and the elimination half-lives (T_{1/2}) was between 7 hours and 9 hours for ezogabine).

Accordingly, an immediate-release formulation of the invention, specifically the immediate-release formulation of Example #12, will provide a therapeutic effect when orally administered in either a fasted or a fed state in the treatment of epilepsy and/or epileptic seizure disorders in a subject, preferably a child, particularly for KCNQ2-related neonatal developmental and epileptic encephalopathy (KCNQ2-DEE).

All of the U.S. patents, U.S. patent application publications, U.S. patent applications, foreign patents, foreign patent applications and non-patent publications referred to in this specification are incorporated herein by reference in their entirety, to the extent not inconsistent with the present description. U.S. Provisional Application No. 62/942,579, filed Dec. 2, 2019, is incorporated herein by reference, in its entirety.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

1. An immediate-release oral pharmaceutical formulation comprising a therapeutically effective amount of ezogabine and one or more pharmaceutically acceptable excipients.

2. The immediate-release oral pharmaceutical formulation of claim 1 comprising two or more pharmaceutically acceptable excipients.

3. The immediate-release oral pharmaceutical formulation of claim 1, wherein each pharmaceutically acceptable excipient is present in a concentration of from about 0.01% w/w to about 99% w/w.

4. The immediate-release oral pharmaceutical formulation of claim 1, wherein ezogabine is present in a concentration of from about 1% w/w to about 30% w/w.

5. The immediate-release oral pharmaceutical formulation of claim 1 comprising ezogabine at a concentration of about 20% w/w; HPMC at a concentration of about 5.0% w/w; starch at a concentration of about 20% w/w; MCC at a concentration of about 45% w/w; butylated hydroxytoluene at a concentration of about 0.01% w/w; and crosprovidone at a concentration of about 10.0% w/w of the immediate-release oral pharmaceutical formulation.

6. A method of treating epilepsy and/or an epileptic seizure disorder in a mammal, wherein the method comprises orally administering to the mammal in need thereof an immediate-release oral pharmaceutical formulation comprising a therapeutically effective amount of ezogabine and one or more pharmaceutically acceptable excipients.

7. The method of claim 6, wherein the mammal is a human.

8. The method of claim 6 wherein the human is a child.

9. The method of claim 6 wherein the epilepsy and/or an epileptic seizure disorder is KCNQ2-related neonatal developmental and epileptic encephalopathy.

10. The method of claim 6 wherein the immediate-release oral pharmaceutical formulation comprises two or more pharmaceutically acceptable excipients.

11. The method of claim 6 wherein each pharmaceutically acceptable excipient is present in a concentration of from about 0.01% w/w to about 99% w/w.

12. The method of claim 6 wherein ezogabine is present in a concentration of from about 1% w/w to about 30% w/w.

13. The method of claim 6 wherein the immediate release oral pharmaceutical formulation comprises ezogabine at a concentration of about 20% w/w; HPMC at a concentration of about 5.0% w/w; starch at a concentration of about 20% w/w; MCC at a concentration of about 45% w/w; butylated hydroxytoluene at a concentration of about 0.01% w/w; and crosprovidone at a concentration of about 10.0% w/w of the immediate-release oral pharmaceutical formulation.

14. A method of preparing an immediate-release oral pharmaceutical formulation comprising comprising a therapeutically effective amount of ezogabine and one or more pharmaceutically acceptable excipients.

15. The method of claim 14 wherein the immediate-release oral pharmaceutical formulation comprises a therapeutically effective amount of ezogabine and two or more pharmaceutically acceptable excipients.

16. The method of claim 4, wherein each pharmaceutically acceptable excipient is present in a concentration of from about 0.01% w/w to about 99% w/w.

17. The method of claim 14, wherein ezogabine is present in a concentration of from about 1% w/w to about 30% w/w.

18. The method of claim 14, wherein the immediate-release formulation comprises ezogabine at a concentration of about 20% w/w; HPMC at a concentration of about 5.0% w/w; starch at a concentration of about 20% w/w; MCC at a concentration of about 45% w/w; butylated hydroxytoluene at a concentration of about 0.01% w/w; and crosprovidone at a concentration of about 10.0% w/w of the immediate-release oral pharmaceutical formulation.
